(19) United States
(12) Patent Application Publication
(10) Pub. No.: US 2024/0408304 A1
(43) Pub. Date: Dec. 12, 2024

(54) DEVICE FOR DELIVERING MEDICAMENT WITH INTEGRATED INFUSION CATHETER AND CONTINOUS GLUCOSE MONITOR SENSOR ASSEMBLY

(71) Applicant: AITA BIO INC., New York, NY (US)

(72) Inventors: Dilan Casanovas Mack, New York, NY (US); Russell Cole, River Vale, NJ (US)

(21) Appl. No.: 18/697,916

(22) PCT Filed: Nov. 10, 2022
(86) PCT No.: PCT/US2022/049564

§ 371 Date: Apr. 2, 2024

Related U.S. Application Data
(60) Provisional application No. 63/279,007, filed on Nov. 12, 2021.

(51) Int. Cl. A61M 5/172; A61M 5/142; A61M 5/158

(52) U.S. Cl. CPC A61M 5/1723; A61M 5/14212; A61M 5/158; A61M 2005/1726; A61M 2230/201

(57) ABSTRACT

A device for delivering medicament to a user is disclosed. The device comprises a reservoir for storing medicament; an infusion catheter fluidly communicating with the reservoir to deliver the medicament into a subcutaneous layer of the user: a pump fluidly communicating with the reservoir and infusion catheter for pumping the medicament from the reservoir though the infusion catheter: an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

### CROSS REFERENCE TO RELATED APPLICATIONS

This application claims priority to U.S. provisional application No. 63/279,007, filed Nov. 12, 2021, entitled “Micropump With Integrated Catheter and Continuous Glucose Monitor Sensor Assembly”, which is incorporated by reference herein.

### FIELD OF THE INVENTION

The present invention relates to a device for delivering medicament with an integrated infusion catheter and continuous glucose monitor sensor assembly.

### BACKGROUND OF THE INVENTION

Current diabetes management systems offer continuous glucose monitoring (CGM) to enable real-time glucose readings of a user. CGM sensors use insertion technology to target the subcutaneous layer of a user's tissue. Fingerstick calibration is required to ensure the sensor is on track. A separate insulin infusion pump is then used for insulin delivery to a user based on the real-time glucose readings. Delivery is typically initiated by the user. This entire system can become very cumbersome. Convenience and discretion are top user needs for insulin pump users. It would thus be advantageous to provide improvements in the current diabetes management systems.

### SUMMARY OF THE INVENTION

A device for delivering medicament with an integrated infusion catheter and continuous glucose monitor sensor assembly is disclosed.

In accordance with an embodiment of the present disclosure, a device for delivering medicament to a user, the device comprising: a reservoir for storing medicament; an infusion catheter fluidly communicating with the reservoir to deliver the medicament into a subcutaneous layer of the user; a pump fluidly communicating with the reservoir and infusion catheter for pumping the medicament from the reservoir though the infusion catheter; an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

In accordance with another embodiment of the disclosure, a device for delivering medicament to a user, the device including a reservoir for storing medicament, a pump fluidly communicating with the reservoir for pumping the medicament from the reservoir into the subcutaneous layer of the user and an assembly for continuously monitoring glucose level in the user and for delivering the medicament to the user based on the monitored glucose level, the assembly comprising: an infusion catheter fluidly communicating with the pump to deliver the medicament into the subcutaneous layer of the user; an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

In accordance with another embodiment of the disclosure, an assembly for continuously monitoring glucose level in a user and for delivering medicament to the user based on the monitored glucose level, the assembly, wherein the assembly is configured to be used with a device for delivering medicament to the user, the device including a reservoir for storing medicament, a pump fluidly communicating with the reservoir for pumping the medicament from the reservoir into the subcutaneous layer of the user and an assembly for continuously monitoring glucose level in the user and for delivering the medicament to the user based on the monitored glucose level, the assembly comprising: an infusion catheter fluidly communicating with the pump to deliver the medicament into the subcutaneous layer of the user; an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

### BRIEF DESCRIPTION OF DRAWINGS

FIG. 1 depicts a perspective view of an integrated infusion catheter and continuous glucose monitoring (CGM) sensor assembly for a micropump (device for delivering medicament) in a pre-activation configuration.

FIG. 2 depicts a perspective view of the assembly in FIG. 1 in a post-activation configuration.

FIG. 3 depicts a perspective view of an example integrated catheter and continuous glucose monitoring (CGM) sensor assembly for a micropump.

FIG. 4 depicts a cross-sectional view of the example assembly in FIG. 3 after insertion into tissue of a user but before the introducer needle is retracted.

FIG. 5 depicts a perspective view of the example assembly in FIG. 3 after the introducer needle has been retracted.

FIG. 6 depicts a perspective view of another example integrated catheter and continuous glucose monitoring (CGM) sensor assembly for a micropump in a pre-activation configuration.

FIG. 7 depicts the example assembly in FIG. 6 in a post activation configuration.

FIG. 8 depicts a perspective view of another example integrated catheter and continuous glucose monitoring (CGM) sensor an alternative sensor and infusion catheter in a post activation configuration, i.e., inserted in the subcutaneous space after introducer needle is retracted.

FIG. 9 depicts a perspective view of another example integrated catheter and continuous glucose monitoring (CGM) assembly with an alternative sensor.

FIG. 10 depicts a perspective view of another example integrated catheter and continuous glucose monitoring (CGM) sensor assembly for a micropump.

FIG. 11 depicts an enlarged view of the assembly in FIG. 10 from line 11-11.

FIGS. 12-15 depicts certain assembly steps for the assembly shown in FIG. 10.

FIGS. 16-25 depict views of other example integrated infusion catheter and continuous glucose monitoring (CGM) sensor assemblies for a micropump.

FIGS. 26-28 depict views of an example assembly including catheter 2602 and introducer needle with a modified introducer needle heal to attach to sensor without welding.

FIG. 29 depicts a block diagram of example components of a micropump (device for delivering medicament to a user).

### DETAILED DESCRIPTION OF THE INVENTION

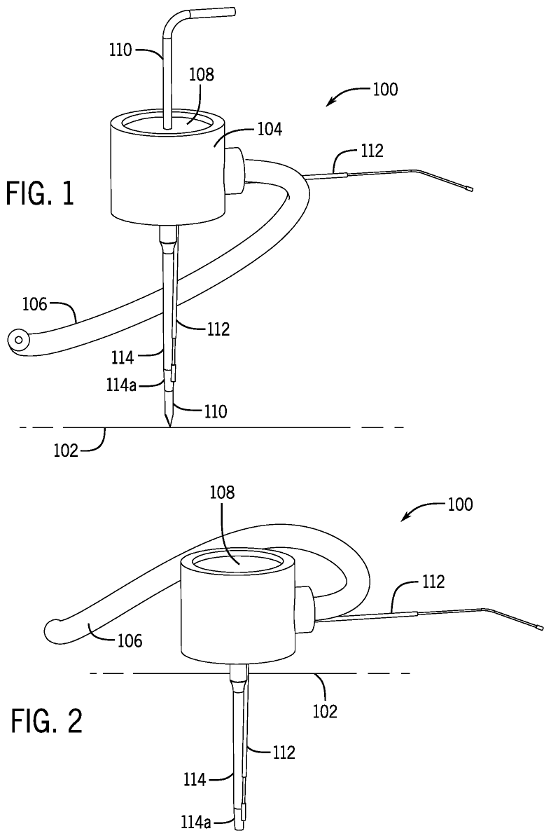

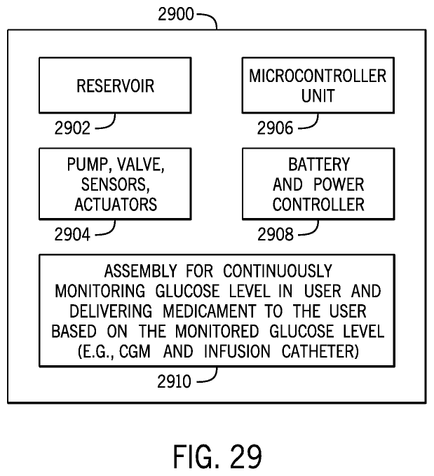

FIGS. 1 and 2 depicts high-level perspective views of integrated infusion catheter and continuous glucose monitoring (CGM) sensor assembly 100 (or device) for micropump 102 (not shown in detail), before and after activation or installation within a user (or patient). Assembly 100 is an example of an assembly for continuous glucose monitoring and delivering medicament to a user based on the monitored glucose level (as shown in FIG. 29 for example). In this respect, CGM and infusion are deployed in a single site configuration.

In this disclosure, micropump 102 is (and also referred to as) a device 102 for delivering medicament (e.g., insulin) to a user (patient) that can be used for pumping fluid, valves used for regulating flow, actuators used for moving or controlling a pumping unit or mechanism or pump and valves and/or sensors used for sensing pressure and/or flow. (However, a micropump may also refer to the pumping mechanism/unit itself such as a MEMS device (as one example)). The device for delivering medicament or micropump may be used in a drug infusion system for infusing a drug (i.e., medication) or other fluid to a patient (user). Medicament may include small molecule pharmaceutical solutions, large molecule or protein drug solutions, saline solutions, blood or other fluids known to those skilled in the art. Insulin is an example of fluid that is described in this application (as the medicament). However, micropump may be used in other environments known to those skilled in the art. Example components of micropump 102 (device for delivering medicament) are shown in FIG. 29 and described below.

The general structure and components of assembly shown in FIGS. 1 and 2 apply to all embodiments described in detail below. Specifically, assembly 100 includes catheter hub 104, insulin tubing 106, septum 108, introducer needle 110, CGM sensor 112 and catheter 114. Assembly 100 also includes a catheter wedge (not shown) within catheter hub 104. Catheter hub 104 receives insulin tubing 106 which communicates with catheter 114 for insulin delivery as known to those skilled in the art.

In FIGS. 1 and 2, and in all other embodiments/examples described hereinbelow, the distal ends of CGM sensor 112 and infusion catheter 114 are fixed relative to one another and move simultaneously downward with introducer needle 110 during insertion in various examples (embodiments) of the configuration, as shown in FIG. 1. (For various embodiments described herein, the CGM sensor may or may not be attached directly to the infusion catheter. In addition, the CGM sensor and infusion catheter may have similar or different lengths therebetween.)

During activation, the proximal end of sensor 112 and insulin tubing 106 bend to maintain electrical connection to a circuit board and fluid connection with the micropump (device for delivering medicament, e.g., insulin). Introducer needle 110 is retracted after insertion and the fluid path is sealed by septum 108 leaving sensor 112 and catheter tip 114a in the subcutaneous tissue layer in the configuration shown in FIG. 2.

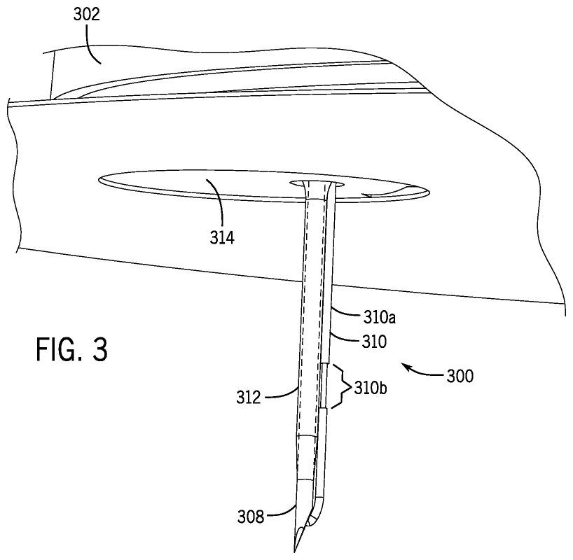

FIG. 3 depicts a perspective view of an example integrated infusion catheter and continuous glucose monitoring (CGM) sensor assembly 300 (or device) for micropump 302 or device 302 for delivering fluid (e.g., insulin). Assembly 300 includes catheter hub 304, catheter wedge 306, introducer needle 308, CGM sensor (wire) 310, catheter 312 and base plate 314. Catheter 312 incorporates lumen 316 for insulin fluid passage and subsequent delivery to a user. Assembly 300 also includes insulin tubing and septum (not shown) that is inserted within a raised edge of catheter hub 304. During assembly, proximal end 312a of catheter 312 is press fit onto the stem of catheter wedge 306. The insulin tubing communicates with catheter 312 for insulin delivery through lumen 316 as known to those skilled in the art.

In this example, sensor 310, is employed to loop around the heal of introducer needle 308. Sensor 310 may be a single long wire or multiple wires (or a component) with or without a sheath. In this respect, assembly 300 avoids having to attach the sensor (wire) 310 to catheter 312 which is a soft plastic and not conducive to coupling with sensor 310. Since both reference electrode 310a and active electrode 310b of the sensor 310 sense in all directions, only a single sensor (wire) is required to loop around introducer needle 308 for integrated insulin infusion and CGM sensing. (Sensor 310 is configured to enable 360 degrees of sensing.) No angular orientation is required between the sensor (wire) 310 and catheter 312. The wire may make line contact with catheter 312 but since most of the wire contacts subcutaneous tissue, the sensor is able to accurately measure blood glucose levels in any angular orientation. No additional hooks, weld material or geometry are necessary to insert sensor 310 into the required depth of the subcutaneous layer. Since sensor wire 310 is looped around introducer needle 308 but not coupled to it, active electrode 310b can move a small amount relative to needle 308 and catheter 312 during insertion but active electrode 310b will be inserted into the depth range required of accurate sensing. During assembly, the distal portion of sensor is inserted into the distal tip of introducer needle and looped around the heal.

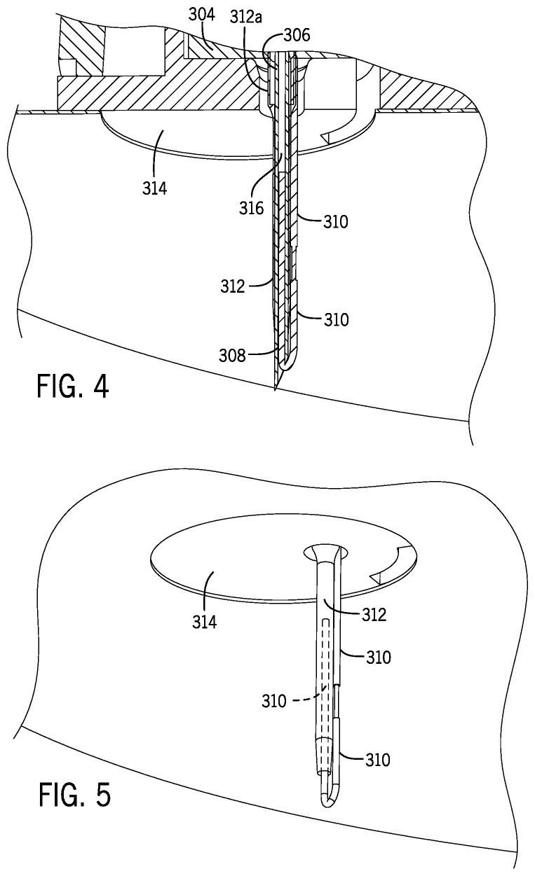

FIG. 4 depicts a cross-sectional view of the example assembly in FIG. 3 after insertion into tissue of a user but before introducer needle 308 is retracted. FIG. 5 depicts a perspective view the example assembly in FIG. 3 in a post-activation configuration, i.e., after the introducer needle has been retracted (as catheter 312 has been inserted in a user).

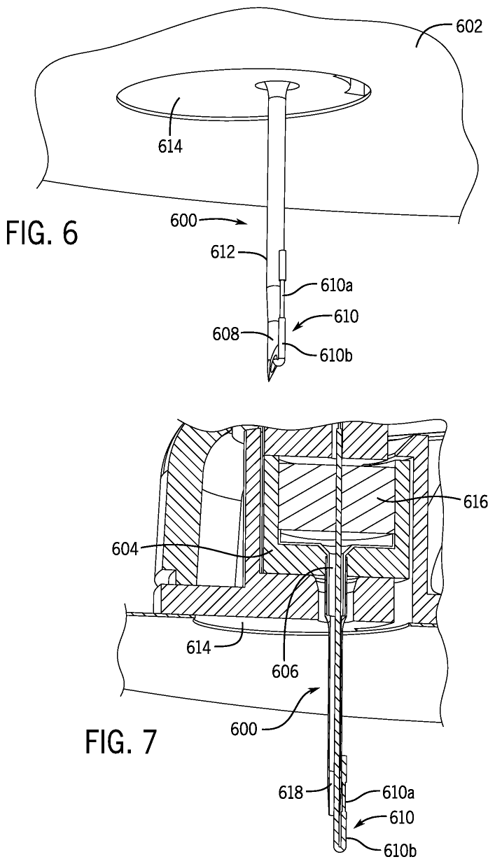

FIG. 6 depicts a perspective view of another example integrated catheter and continuous glucose monitoring (CGM) sensor assembly 600 (or device) for device 602 for delivering medicament or micropump 602. The medicament may be insulin for example or another medicament as known to those skilled in the art. Assembly 600 includes catheter hub 604, catheter wedge 606, introducer needle 608, CGM sensor (wire) 610, catheter 612, base plate 614 and septum 616. Catheter 612 incorporates lumen 618 for insulin fluid passage and subsequent delivery to a user. Assembly 600 also includes insulin tubing (not shown) that is inserted within a raised opening in catheter hub 604.

In this example, CGM sensor 610, with a circular cross section, loops around the heal of introducer needle 608, the tip of catheter 612 or an opening therein toward the distal end. The difference between this example assembly 600 and the assembly in FIGS. 3-5 is that the proximal end in this example assembly 600 extends through the center of catheter 612 and the distal end loops around the tip and resides above the catheter 612 tip in the subcutaneous space next to but not attached to infusion catheter 612 as shown. Active electrode 610a and reference electrode 610b of sensor 610 are also shown. In this configuration, sensor 610 inserts into a user's tissue with no additional hooks or attachment features that make the inserted components larger and more painful. Assembly 600 contains sensor 610 in lumen 618 of introducer needle 608 so needle 608 would need to be C-shaped in order to retract and detach from the assembly. Alternately, introducer needle 608 could be a standard introducer needle but must remain in the micropump 602 housing after retraction by bending over after retraction to stow in place. The non-user (patient) end exits the fluid path to connect to a printed circuit board (PCB) through septum 616 as shown in the post-activation cross section in FIG. 7 or through another sealed interface.

Alternately, sensor 610 can be placed between catheter 612 and the outside of introducer needle 608 and loop around the catheter tip which depicts the device after insertion into the tissue but before introducer needle 608 is retracted. The advantage of this example is that sensor 610 is outside of introducer needle lumen 618 so introducer needle 608 can be easily retracted from the assembly.

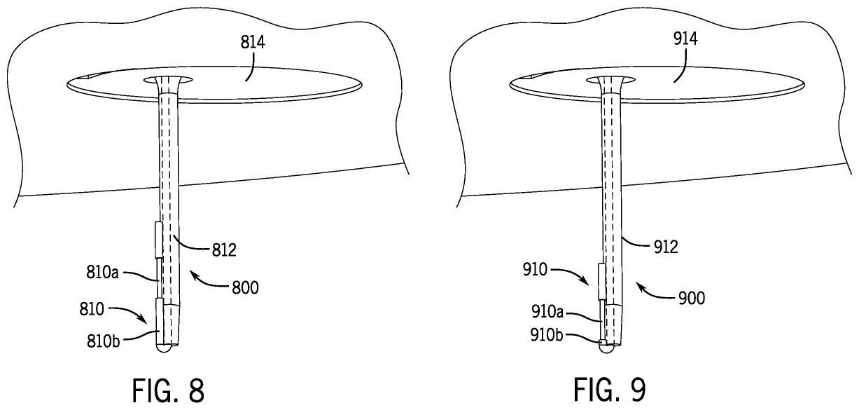

FIG. 8 depicts a perspective view of another example integrated catheter and continuous glucose monitoring (CGM) sensor assembly 800 with an alternative sensor 810 and infusion catheter 812 in a post activation configuration, i.e., inserted in the subcutaneous space after introducer needle is retracted. Otherwise, assembly 800 has similar components as described above with respect to FIGS. 6-7 (e.g., base plate 814). In this example, sensor 810 exits through distal end of catheter 812 and active electrode 810a is farther from the opening of catheter 812 and closer to the surface of the skin/tissue of the user in order to keep active portion farther from insulin deposition. (Reference electrode 810b is also shown.) Insulin is an example of medicament fluid.

FIG. 9 depicts a perspective view of another example assembly 900 with an alternative sensor 910. In this respect, active electrode 910a of sensor 910 is closer to the opening of catheter 912. Otherwise, assembly 800 has similar components as described above with respect to FIGS. 6-7 (e.g., base plate 914). (Reference electrode 910b is also shown.) Other medicaments may be used as known to those skilled in the art.)

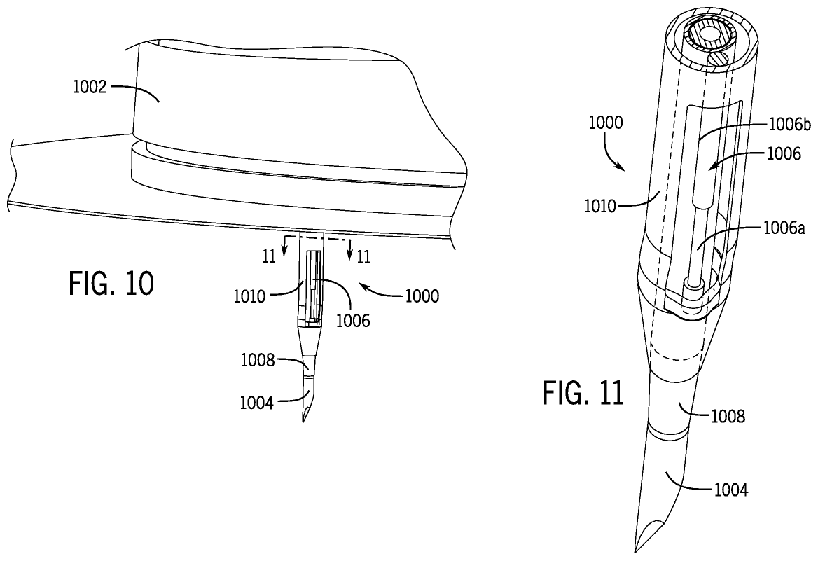

FIG. 10 depicts a perspective view of another example integrated catheter and continuous glucose monitoring (CGM) sensor assembly 1000 (or device) for device 1002 for delivering medicament (e.g., insulin) or micropump 1002. Similar to the other examples described herein, assembly 1000 includes introducer needle 1004, CGM sensor 1006, insulin catheter 1008, sheath catheter 1010, catheter hub 1012, catheter wedge 1014, insulin tubing and septum but these two components are not shown here. The catheter hub receives the insulin tubing 106 through a port which communicates with catheter 1008 for insulin delivery as known to those skilled in the art. Sheath catheter 1010 is configured to hold sensor 1006 as shown. Sheath catheter 1010 incorporates a side (laser cut) window to enable access to interstitial fluid by active electrode 1006a and reference electrode 1006b (FIG. 11).

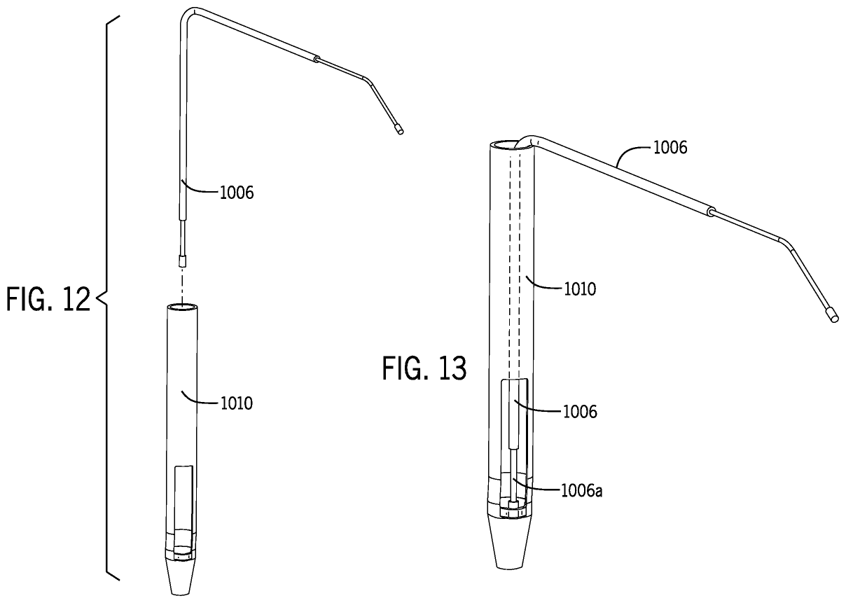

One of the primary difficulties with manufacturing a wire sensor based integrated catheter is assembling the wire sensor into any other component because the wire is small (the diameter is in the ballpark of one to two human hairs) and flexible. Assembly of these components avoids this challenge by loosely placing CGM sensor 1006 into the sheath catheter 1010 as shown in FIGS. 12 and 13.

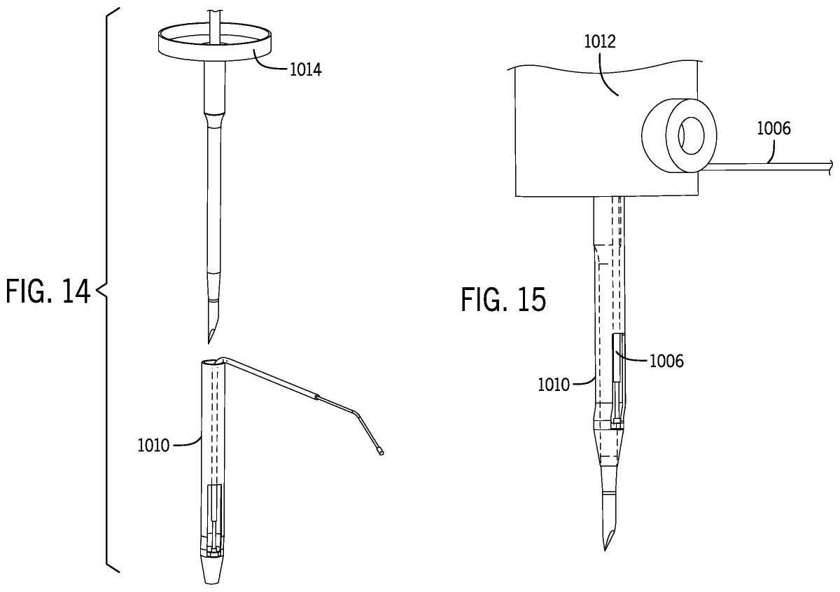

In the next assembly step as shown in FIGS. 14 and 15, a standard fluid path catheter 1008, wedge (not shown) and introducer needle assembly are inserted inside the sheath catheter 1010 trapping wire sensor 1006 between the outside of the infusion catheter and inside of the sheath catheter. This holds sensor 1006 in place allowing it to be inserted into the subcutaneous space along with insulin catheter 1008 using a single insertion mechanism and introducer needle 1004.

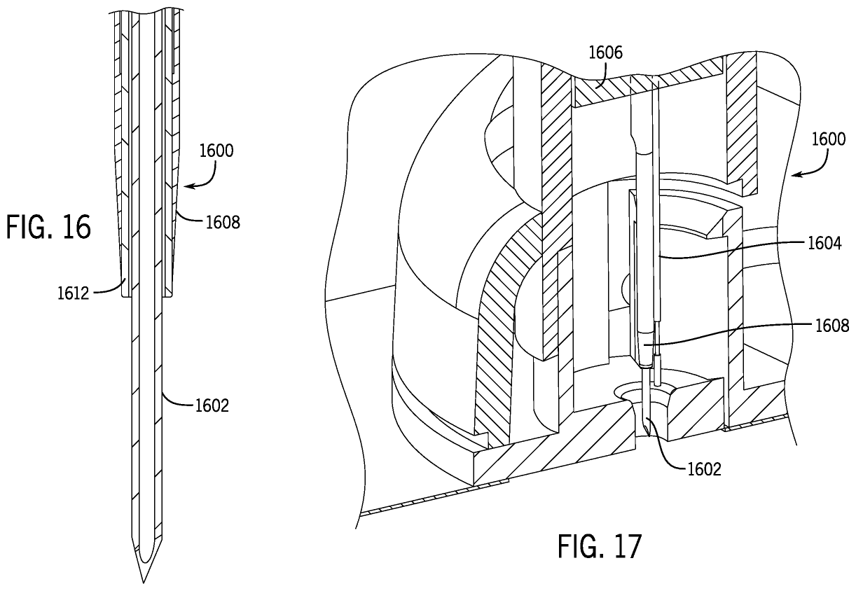

FIGS. 16-25 depict various views of other example integrated catheter and continuous glucose monitoring (CGM) sensor assemblies (or devices) for a micropump or a device for delivering medicament (e.g., insulin). Referring to FIGS. 16-22, as in other assemblies described herein, assembly 1600 includes introducer needle 1602, CGM sensor (wire) 1604, catheter hub 1606, infusion catheter 1608, base plate 1610 (Assembly 1600 also includes a catheter wedge and septum (not shown). Catheter 1608 incorporates a lumen for insulin fluid passage and subsequent delivery to a user. Assembly 1600 also includes insulin tubing (not shown) that is inserted within a raised edge of catheter hub 1606.

Assembly 1600 provides a sensor/catheter integration solution that is as close to outer diameter of a typical insulin infusion catheter 1608 as possible to minimize insertion pain. Catheter 1608 and introducer needle 1602 configuration in this example is similar to that of all insulin infusing micropump examples described herein in that the introducer needle is inside of a plastic infusion catheter except however in this example, assembly 1600 also includes a steel tube 1612 between catheter 1608 and introducer needle 1602. This provides the rigidity necessary to detach sensor 1604 from introducer needle 1602 when it retracts after insertion into the tissues.

In this example, CGM (wire) sensor 1604 is unattached and free floating on the outside of catheter 1608 as shown in the pre-activation configuration (FIG. 17). The distal tip of sensor 1604 is welded to introducer needle 1602 so that when needle 1602 inserts into the tissue the sensor 1604 is also inserted.

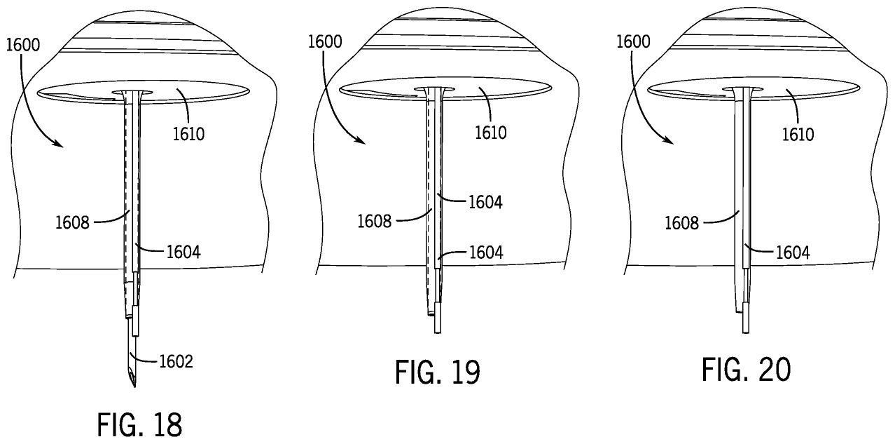

FIG. 18 depicts a configuration of assembly 1600 after insertion into the tissue but before introducer needle 1602 is retracted. Sensor (wire) 1604 is still welded or attached by other means at this point. FIG. 19 depicts a configuration of assembly 1600 after introducer needle 1602 is retracted. The blunt tip tubing 1612 inside the plastic infusion catheter 1608 provides the rigid body required to break the attachment between the wire sensor 1604 and introducer needle 1602. The blunt tip needle could remain in the body inside of infusion catheter 1608 or could retract after introducer needle 1602 retracts leaving just sensor 1604 and infusion catheter 1608 in the tissue as shown in FIG. 20.

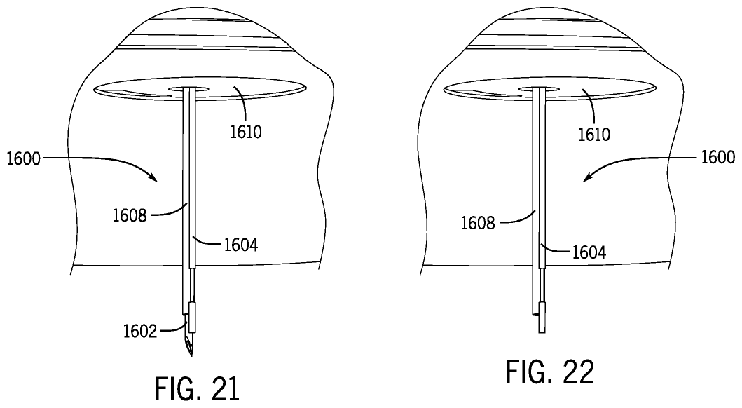

An alternative example, plastic infusion catheter may be eliminated as shown in FIGS. 21 and 22. The steel in dwelling blunt tip tube 1612 remains in the body after insertion and needle retraction (FIG. 22). Removing the sharp tip will reduce pain and minimize wound healing response.

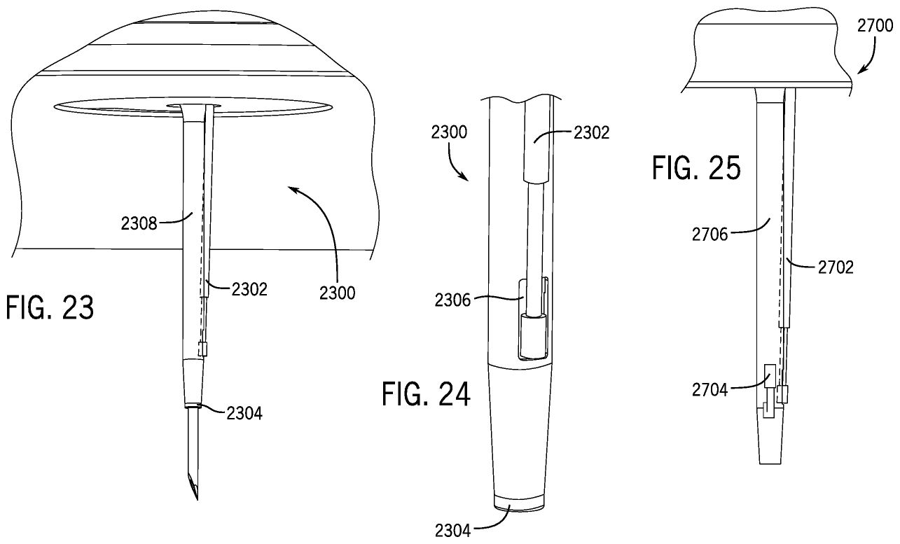

Another alternative example of the assembly 2300 appears in FIGS. 23 and 24. The benefit of this alterative example is that sensor 2302 is attached to the blunt tip tube 2304 through a window 2306 in plastic catheter 2308 so that the sensor is farther away from the insulin deposition reducing the probability of interference between the insulin and sensor electrodes.

The example assembly 2700 in FIG. 25 is similar to the assembly 2300 in FIGS. 23 and 24 except a small portion of stainless steel tube 2704 is assembled into the distal end providing an attachment surface for the tip of sensor 2702. The tube section 2704 remains in plastic catheter 2706 because the tipping process reduces the inner diameter of catheter 2706 such that the steel cannot come out.

The example assemblies in FIGS. 16-25 rely on welding or attaching the sensor to a blunt tip tube. For most of these example assemblies the attachment is strong enough to hold the wire during insertion but weak enough to break when the introducer needle retracts. The weld will not add material that can dislodge in the body or prevent the introducer needle from retracting through the blunt tip tubing.

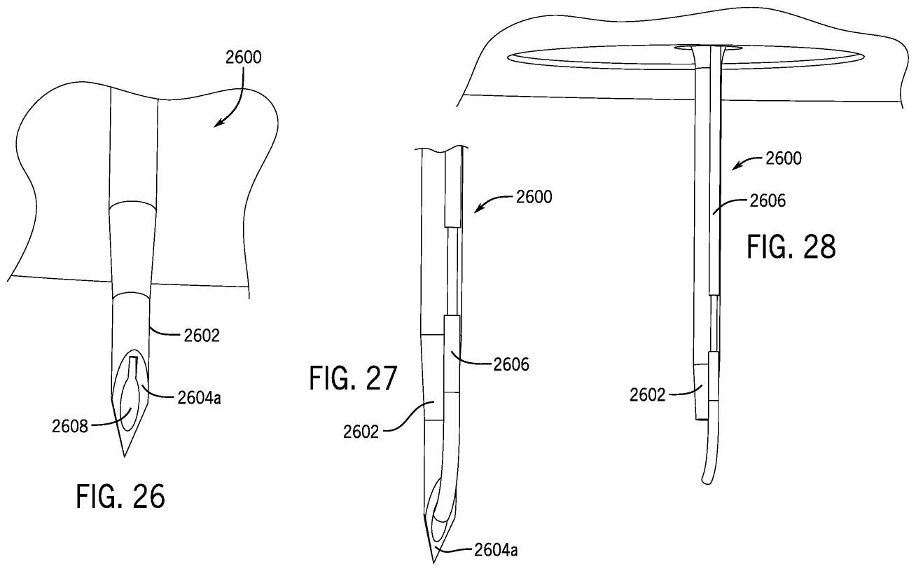

FIGS. 26-28 depict various views of example assembly 2600 including infusion catheter 2602 and introducer needle 2604 with a modified introducer needle heal 2604a to attach to sensor 2604 without welding. (FIG. 27 does not show sensor 2606.) A slot 2608 is laser cut into the heal 2604 and the inactive portion of the sensor 2606 is press-fit into slot 2608 as shown.

When needle 2604 moves in the direction of insertion slot 2608 maintains attachment to sensor 2606 allowing sensor 2606 to insert with needle 2604. When needle 2604 retracts, sensor 2606 is pulled out of the slot thereby detaching needle 2604 and sensor 2606. The configuration in FIG. 28 depicts catheter 2602 and sensor 2606 after insertion and needle retraction.

In an alternative example of the assembly above, the CGM sensor exits through the side of the catheter so the catheter tip is in a configuration for tissue penetration with the catheter tip tapered and press fit around the introducer needle.

FIG. 29 depicts several components of micropump 2900 or device 2900 for delivery medicament or to a user. Micropump 2900 is similar (with same components) as those example micropumps described hereinabove (but renumbered). In this configuration, the device includes reservoir 2902, pumping unit 2904, microcontroller unit (MCU) 2906, battery and power controller 2908 and integrated infusion catheter and glucose monitoring assembly 2910 as described in detail above with respect to all embodiments. MCU 2906 controls the operation of pumping unit 2904.

Reservoir 2902 is configured to receive and store medicament such as insulin for its delivery over a course of about three days, or as needed. However, reservoir size may be configured for storing any quantity of fluid as required.

Pumping mechanism or pumping unit 2904 fluidly communicates with reservoir 2902 to enable infusion as needed. Pumping unit 2904 includes one or more pumps, values sensors and/or actuators as known to those skilled in the art. In one example configuration, pumping unit 2904 may connect directly to reservoir 2902. In another example configuration, a short interposer may be used as a connector. Pumping unit 2904 also fluidly communicates with an infusion catheter of assembly 2910 for insulin delivery. An infusion catheter

Pumping unit 2904 may incorporate one or more MEMS devices (micro-electro-mechanical systems as known to those skilled in the art), for example, that function as a pump for pumping fluid such as insulin, valves for regulating flow, actuators for moving or controlling the pump and valves, and sensors for sensing pressure, insulin flow, presence of air in the fluid path and across the channels in the MEMS devices. In one example configuration, the MEMS devices are each a piezoelectric transducer (or other MEMS devices including capacitive transducers or piezoresistive transducers) that acts as the active element for pumping fluid, but other MEMS structures or technology may be used to achieve desired results as known to those skilled in the art. Operation and functional details of the MEMS devices (e.g., piezoelectric transducer) appear in more detail below. Pumping unit 2904 however may be any pumping mechanism other than a MEMS device as known to those skilled in the art that functions similarly as needed.

Battery and power controller 2908 controls the power to MCU 2906 and pumping unit 2904 to enable those components to function properly as known to those skilled in the art. The CGM sensor may be powered by battery and power controller 2908 through MCU 2906.

It is to be understood that the disclosure teaches examples of the illustrative embodiments and that many variations of the invention can easily be devised by those skilled in the art after reading this disclosure and that the scope of the present invention is to be determined by the claims below.

1. A device for delivering medicament to a user, the device comprising:
a reservoir for storing medicament;
an infusion catheter fluidly communicating with the reservoir to deliver the medicament into a subcutaneous layer of the user;
a pump fluidly communicating with the reservoir and infusion catheter for pumping the medicament from the reservoir though the infusion catheter;
an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and
a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

2. The device of claim 1 wherein the sensor and infusion catheter each have distal ends that are fixed relative to one another.

3. The device of claim 1 further comprising a catheter sheath around the infusion catheter, wherein the sensor is positioned between the infusion catheter and catheter sheath thereby holding the sensor in place.

4. The device of claim 3 wherein the catheter sheath includes an opening to enable an active electrode of the sensor to access an interstitial fluid of the user after insertion.

5. The device of claim 1 wherein the sensor extends along an exterior of the infusion catheter for simultaneous insertion of the sensor and infusion catheter.

6. The device of claim 1 wherein the sensor is attached to the introducer needle for simultaneous insertion with the infusion catheter, wherein the sensor is configured to detach from the introducer needle when the introducer needle is retracted.

7. The device of claim 1 further comprising a rigid tube positioned between the introducer needle and infusion catheter.

8. The device of claim 7 wherein the infusion catheter includes an opening exposing a section of the rigid tube to the interstitial fluid of the user, wherein the sensor extends along an exterior of the infusion catheter and includes a distal end that is attached to section of the rigid tube.

9. The device of claim 7 wherein the rigid tube extends from a distal end of the infusion catheter, wherein the sensor extends along an exterior of the infusion catheter and includes a distal end that is attached to the distal end of the rigid tube.

10. The device of claim 1 wherein the sensor configured to extend around an exterior of the infusion catheter and loop around a heal of the introducer needle, wherein the sensor includes an active electrode exposed to the interstitial fluid of the user, thereby enabling sensing.

11. The device of claim 1 wherein the sensor extends through a lumen of the introducer needle with a distal end of the sensor looping around a heal of the introducer needle or a tip of the infusion catheter and residing on the tip in the subcutaneous space adjacent the infusion catheter.

12. The device of claim 9 wherein the sensor includes an active electrode and reference electrode, the active electrode being either adjacent to the tip of the infusion catheter or spaced a distance from the tip along the outside of the infusion catheter.

13. A device for delivering medicament to a user, the device including a reservoir for storing medicament, a pump fluidly communicating with the reservoir for pumping the medicament from the reservoir into the subcutaneous layer of the user and an assembly for continuously monitoring glucose level in the user and for delivering the medicament to the user based on the monitored glucose level, the assembly comprising:
an infusion catheter fluidly communicating with the pump to deliver the medicament into the subcutaneous layer of the user;
an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and
a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

14. The device of claim 13 wherein the sensor and infusion catheter each have distal ends that are fixed relative to one another.

15. The device of claim 13 wherein the assembly further comprising a catheter sheath around the infusion catheter, wherein the sensor is positioned between the infusion catheter and catheter sheath thereby holding the sensor in place.

16. The device of claim 15 wherein the catheter sheath includes an opening to enable an active electrode of the sensor to access an interstitial fluid of the user after insertion.

17. The device of claim 13 wherein the sensor extends along an exterior of the infusion catheter for simultaneous insertion of the sensor and infusion catheter.

18. The device of claim 13 wherein the sensor is attached to the introducer needle for simultaneous insertion with the infusion catheter, wherein the sensor is configured to detach from the introducer needle when the introducer needle is retracted.

19. The device of claim 13 wherein the assembly further comprising a rigid tube positioned between the introducer needle and infusion catheter.

20. The device of claim 19 wherein the infusion catheter includes an opening exposing a section of the rigid tube to the interstitial fluid of the user, wherein the sensor extends along an exterior of the infusion catheter and includes a distal end that is attached to section of the rigid tube.

21. The device of claim 19 wherein the rigid tube extends from a distal end of the infusion catheter, wherein the sensor extends along an exterior of the infusion catheter and includes a distal end that is attached to the distal end of the rigid tube.

22. The device of claim 13 wherein the sensor is configured to extend around an exterior of the infusion catheter and loop around a heal of the introducer needle, wherein the sensor includes an active electrode exposed to the interstitial fluid of the user, thereby enabling sensing.

23. The device of claim 13 wherein the sensor extends through a lumen of the introducer needle with a distal end of the sensor looping around a heal of the introducer needle or a tip of the infusion catheter and residing on the tip in the subcutaneous space adjacent the infusion catheter.

24. The device of claim 21 wherein the sensor includes an active electrode and reference electrode, the active electrode being either adjacent to the tip of the infusion catheter or spaced a distance from the tip along the outside of the infusion catheter.

25. An assembly for continuously monitoring glucose level in a user and for delivering medicament to the user based on the monitored glucose level, wherein the assembly is configured to be used with a device for delivering medicament to the user, the device including a reservoir for storing medicament, a pump fluidly communicating with the reservoir for pumping the medicament from the reservoir into the subcutaneous layer of the user and an assembly for continuously monitoring glucose level in the user and for delivering the medicament to the user based on the monitored glucose level, the assembly comprising:
an infusion catheter fluidly communicating with the pump to deliver the medicament into the subcutaneous layer of the user;
an introducer needle movable within the infusion catheter, the introducer needle configured to facilitate (a) insertion of the infusion catheter into a subcutaneous layer of the user and (b) retraction of the introducer needle after insertion of the infusion catheter to enable delivery of medicament through the infusion catheter; and
a sensor for continuously monitoring glucose level in the user, wherein the sensor is configured to (a) engage the introducer needle and/or the infusion catheter and (b) simultaneously advance along with the infusion catheter during the insertion into the subcutaneous layer of the user by the introducer needle.

26. The device of claim 25 wherein the sensor and infusion catheter each have distal ends that are fixed relative to one another.

27. The device of claim 25 wherein the assembly further comprising a catheter sheath around the infusion catheter, wherein the sensor is positioned between the infusion catheter and catheter sheath thereby holding the sensor in place.

28. The device of claim 27 wherein the catheter sheath includes an opening to enable an active electrode of the sensor to access an interstitial fluid of the user after insertion.

29. The device of claim 13 wherein the sensor extends along an exterior of the infusion catheter for simultaneous insertion of the sensor and infusion catheter.
